(19) United States
(12) Patent Application Publication
(10) Pub. No.: US 2016/0338620 A1
(43) Pub. Date: Nov. 24, 2016

(54) PORTABLE BREATHALYZER DEVICE

(71) Applicant: Shaun Masavage, Arlington, VA (US)

(72) Inventor: Shaun Masavage, Arlington, VA (US)

(21) Appl. No.: 15/138,131

(22) Filed: Apr. 25, 2016

Related U.S. Application Data
(60) Provisional application No. 62/152,233, filed on Apr. 24, 2015.

(51) Int. Cl. A61B 5/097; A61B 5/08

(52) U.S. Cl. CPC A61B 5/097; A61B 5/082; A61B 2010/0009

(57) ABSTRACT

A portable blood alcohol sensing device has an enclosure having an end with at least one vent opening defined therein. A printed circuit board assembly is disposed within the enclosure, with a connector at one end and a sensing device at the other. An air flow device with a diverter and ramp is disposed is disposed within the enclosure adjacent to the vent openings. The ramp includes an opening in which a body portion of the sensor is disposed. The ramp is disposed substantially horizontally at an upward angle relative to the printed circuit board assembly and intake vent openings. The diverter is disposed substantially longitudinally and an inward angle relative to the at least one vent opening. The intake vent openings, the diverter and the ramp define an airflow path through an interior of the enclosure, along a side and over a top portion of the sensing device.

### BACKGROUND

1. Field

The aspects of the disclosed embodiments generally relates to breathalyzer devices, and more particularly to a portable breathalyzer device.

2. Description of Related Developments

The monitoring of breath alcohol content is important, and particularly so when performing certain activities such as operating machinery and driving. Having a quantified analysis of one's blood alcohol content (BAC) can be useful in determining whether to operate machinery, drive a vehicle or make other decisions where the understanding and regulation of BAC is important.

To date, breathalyzer devices that are used to measure BAC tend to be large and bulky. It would be advantageous to provide a small sized, portable breathalyzer BAC measurement device that overcomes the drawbacks of the prior art.

Accordingly, it would be desirable to provide a portable breathalyzer device that addresses at least some of the problems identified above.

### SUMMARY

The aspects of the disclosed embodiments provide a portable breathalyzer device, as is recited by the subject matter of the independent claims. Further advantageous modifications can be found in the dependent claims.

According to a first aspect, the disclosed embodiments are directed toward a portable blood alcohol sensing device. In one embodiment, the portable blood alcohol sensing device comprises an enclosure having an end with at least one vent opening defined therein. A printed circuit board assembly is disposed within the enclosure, the printed circuit board assembly having a connector at one end and a sensing device at an other end. An air flow device disposed is disposed within the enclosure adjacent to the at least one vent opening, the air flow device including a diverter portion and a ramp portion. The ramp portion includes an opening, a body portion of the sensor configured to be disposed within the opening in the ramp portion. The ramp portion is disposed substantially horizontally at an upward angle relative to the printed circuit board assembly and intake vent openings. The diverter portion is disposed substantially longitudinally and an inward angle relative to the at least one vent opening. The at least one intake vent opening, the diverter portion and the ramp portion define an airflow path through an interior of the enclosure, along a side and over a top portion of the sensing device.

These and other aspects, implementation forms, and advantages of the exemplary embodiments will become apparent from the embodiments described herein considered in conjunction with the accompanying drawings. It is to be understood, however, that the description and drawings are designed solely for purposes of illustration and not as a definition of the limits of the disclosed invention, for which reference should be made to the appended claims. Additional aspects and advantages of the invention will be set forth in the description that follows, and in part will be obvious from the description, or may be learned by practice of the invention. Moreover, the aspects and advantages of the invention may be realized and obtained by means of the instrumentalities and combinations particularly pointed out in the appended claims.

### BRIEF DESCRIPTION OF THE DRAWINGS

In the following, the invention will be explained in more detail with reference to the example embodiments shown in the drawings, in which:

FIG. 1 is a perspective view of a portable breathalyzer device incorporating aspects of the disclosed embodiments;

FIG. 2 illustrates a side view of the device shown in FIG. 1.

FIG. 3 illustrates a cap end view of the device shown in FIG. 1.

FIG. 4 illustrates a top view of the device shown in FIG. 1.

FIG. 5 is a perspective view of a portable breathalyzer device incorporating aspects of the disclosed embodiments, with the top portion in phantom lines.

FIG. 6 illustrates a side view of the device shown in FIG. 5.

FIG. 7 illustrates a cap end view of the device shown in FIG. 5.

FIG. 8 illustrates a top view of the device shown in FIG. 5.

FIG. 9 is a perspective view of a portable breathalyzer device incorporating aspects of the disclosed embodiments, with the top portion in phantom lines and the end cap removed;

FIG. 10 illustrates a side view of the assembly shown in FIG. 9.

FIG. 11 illustrates an end view of the assembly shown in FIG. 9.

FIG. 12 illustrates a top view of the assembly shown in FIG. 9.

FIG. 13 is a perspective view of a portable breathalyzer device incorporating aspects of the disclosed embodiments, with the top portion and end cap removed;

FIG. 14 illustrates a side view of the assembly shown in FIG. 13.

FIG. 15 illustrates a end view of the assembly shown in FIG. 13.

FIG. 16 illustrates a top view of the assembly shown in FIG. 13.

FIG. 17 illustrates the PCB assembly, sensor and air flow device for the device shown in FIG. 1.

FIG. 18 illustrates a side view of the assembly shown in FIG. 17.

FIG. 19 illustrates an end view of the assembly shown in FIG. 17.

FIG. 20 illustrates a top view of the assembly shown in FIG. 17.

FIG. 21 illustrates the PCB assembly and sensor for the device shown in FIG. 1.

FIG. 22 illustrates a side view of the assembly shown in FIG. 21.

FIG. 23 illustrates an end view of the assembly shown in FIG. 21.

FIG. 23 illustrates a top view of the assembly shown in FIG. 21.

FIG. 25 is an assembly view of the device shown in FIG. 1.

FIG. 26 illustrates the airflow path through the device shown in FIG. 1.

FIG. 27 illustrates the device of the disclosed embodiments coupled to a mobile device.

FIGS. 28 and 29A-29D illustrate screen shots of application used with the device of the disclosed embodiments.

FIG. 30 is an article describing use, benefits and advantages of the device.

### DESCRIPTION OF THE DISCLOSED EMBODIMENTS

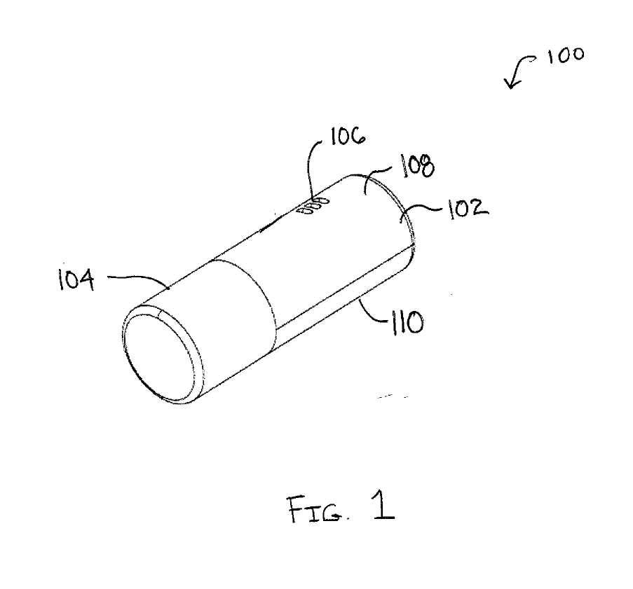

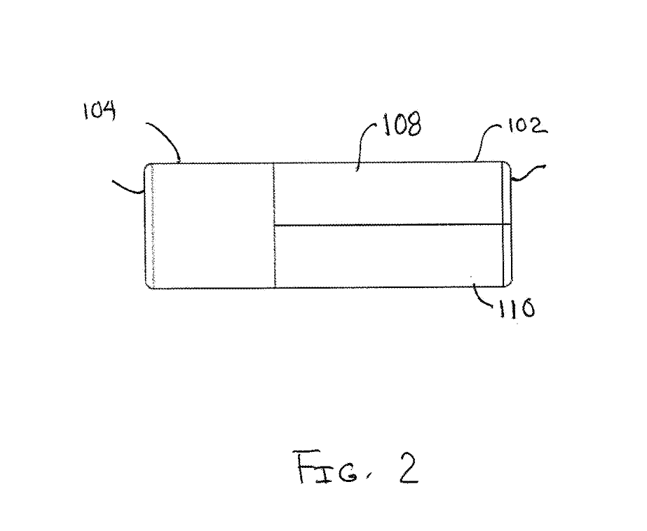

FIG. 1 illustrates a perspective view of a breathalyser device incorporating aspects of the disclosed embodiments. The aspects of the disclosed embodiments are directed to a portable breathalyzer device that can be connected to a computing device such as a smartphone and used to measure and display blood alcohol content in a manner that is readily understood. This can include a relatively precise number, range or other indication of the blood alcohol content (“BAC”). The breathalyzer device of the disclosed embodiments provides a small form factor, cost reduction, battery elimination, and no requirement to bring the device in physical content (touch) with the user's mouth. This advantageously can provide a reduction in the spread of germs/diseases, and improve the multi-use facets of the device 100. The overall design of the breathalyzer device of the disclosed embodiments is much more convenient than traditional breathalyzers and easier to carry around.

As is shown in FIG. 1, the device 100 generally comprises an enclosure or body portion 102 and a cap portion 104. In the example of FIG. 1, a plurality of exhaust ports 106, as will be described further herein, are provided in a sidewall of a portion 108 of the body portion 102. For the purposes of the disclosure herein, the portion 108 will be referred to as the upper portion 108. The body portion 102 will include a lower potion 110. The upper portion 108 and bottom portion 110 are configured to be mated together as will be described herein.

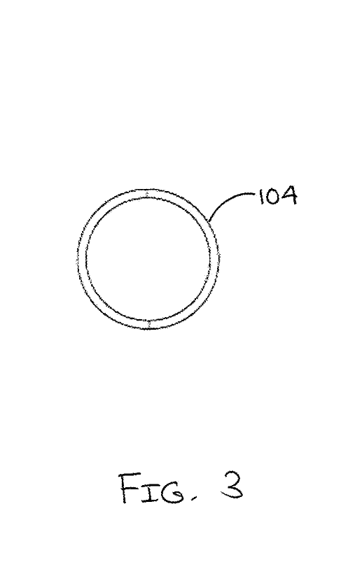

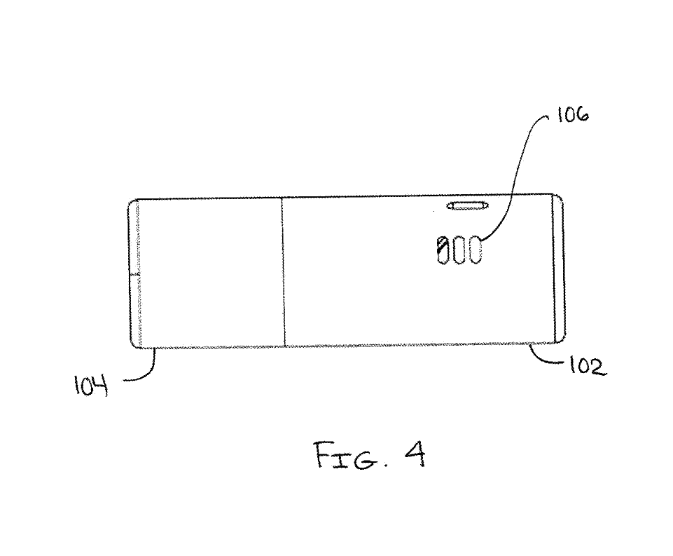

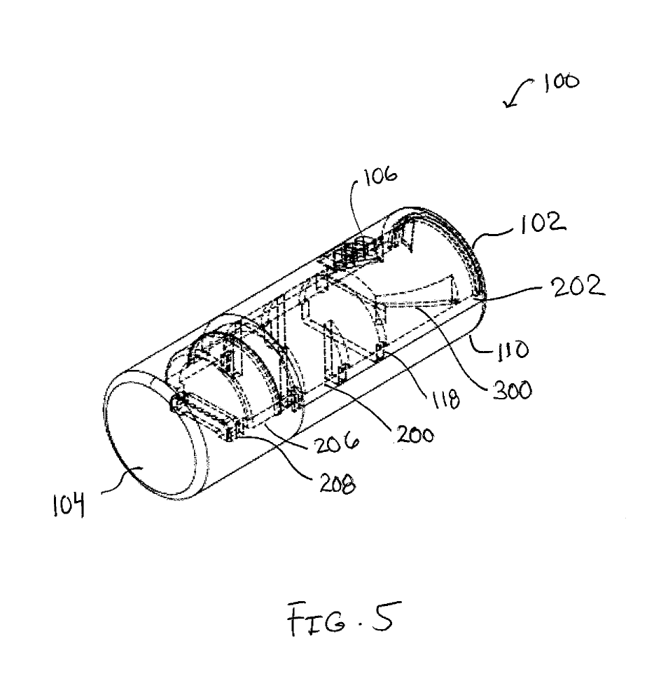

FIGS. 2-4 illustrate a side view, an end view, and a top view of the device 100 shown in FIG. 1. The enclosure 102 of the device 100 shown in FIGS. 1-4 is generally cylindrical in shape. In alternate embodiments, the shape of the enclosure 102 of the device 100 can be any suitable geometric shape that can be used to provide the functions and advantages described herein. For example, in one embodiment the enclosure 102 of the device 100 could have a rectangular form or shape, where a dimension of the length is greater than a dimension of the height.

In one embodiment, the device 100 of the disclosed embodiments has a shape and a size that is approximately 1.9″ long×0.6″ in diameter. In the example of FIG. 1 the dimensions of the device 100, comprising the enclosure portion 102 and cap portion 104 are approximately 0.6″×0.6″×1.9″ (16 mm×16 mm×48 mm). However, in alternate embodiments, as noted above, the device 100 can comprise any suitable size that provides the portable functionality generally described herein.

In one embodiment, the device 100 weighs only about 0.25 oz (7.0 g). While other sizes and weights are within the scope of contemplation, the device 100 of the disclosed embodiments is generally intended to be small and portable. Thus, the device 100 of the disclosed embodiments can have any suitable size, shape and weight that achieves such a portable configuration.

In one embodiment, the device 100 is configured so that the center of weight of the device 100 is offset towards the bottom portion 110 of the device 100. When the device 100 has a cylindrical shape such as that shown in FIG. 1, weighting the bottom portion 110 can prevent the device 100 from rolling, such as rolling off of a surface that is not completely flat. In this embodiment, the weight towards the bottom portion 110 of the device 100 essentially acts as a ballast. For example, the PCB board assembly 200 can be positioned lower in the bottom portion 110 relative to the top portion 108 to create this weighting and ballast.

The material of the device 100, such as the body portion 102 and cap portion 104 can generally comprise a plastic material, such as an ABS plastic. In alternate embodiments, any suitable material can be used. The device 100 can also have any suitable color and finish. Some example of finishes can include, but are not limited to, rubberized matte, chrome or paper with different graphics (patterns, wood-patterned, etc.)

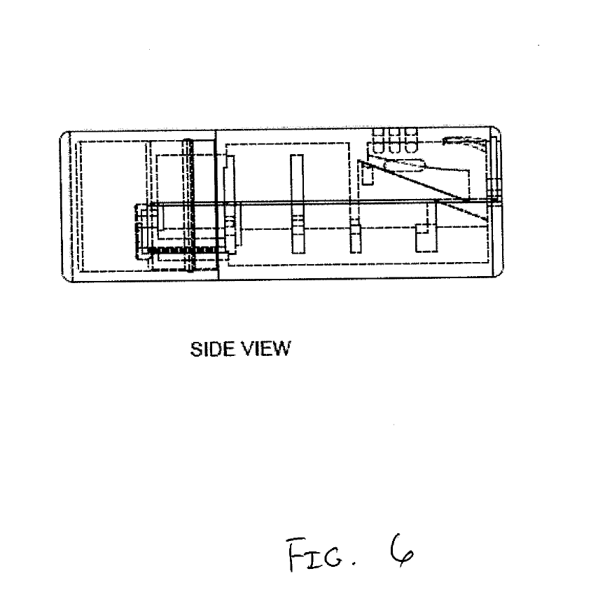

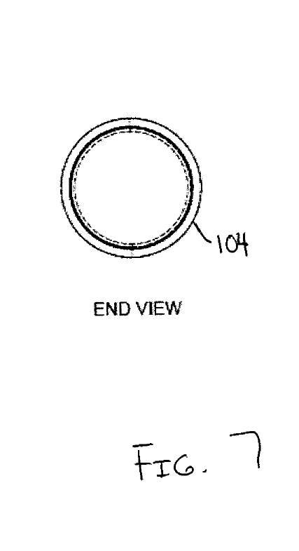

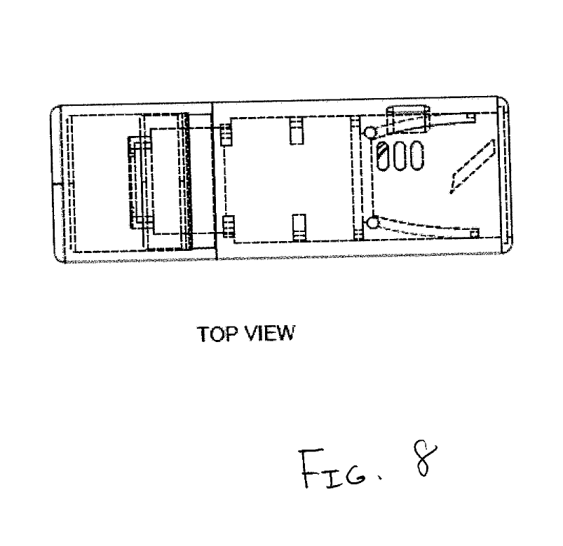

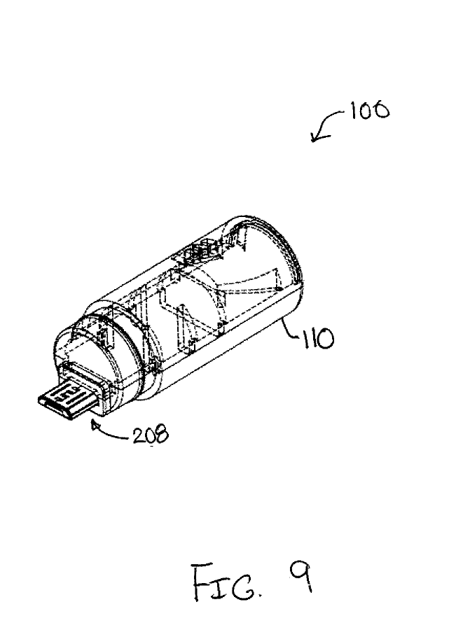

FIGS. 5-8 illustrate the device 100 of FIG. 1 with portions of the top 108 to expose the interior construction of the device 100. In this example, the outline of the printed circuit board assembly 200 is visible. One end 202 of the circuit board assembly 200 includes an airflow device 300. An other end 206 of the printed circuit board assembly 200 includes a connector portion 208. The connector portion 208 is covered by the cap 104. In one embodiment, the airflow device 300 is generally proximate the openings or vents 106 illustrated in FIG. 1.

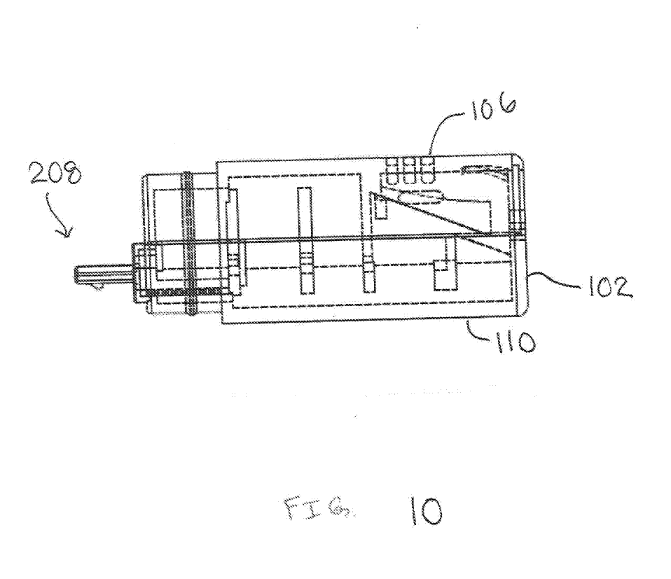

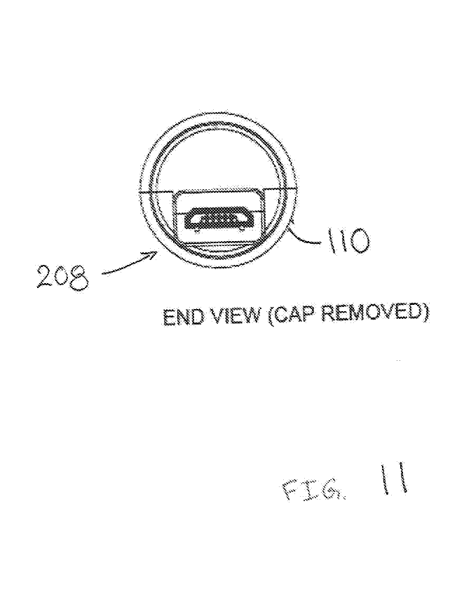

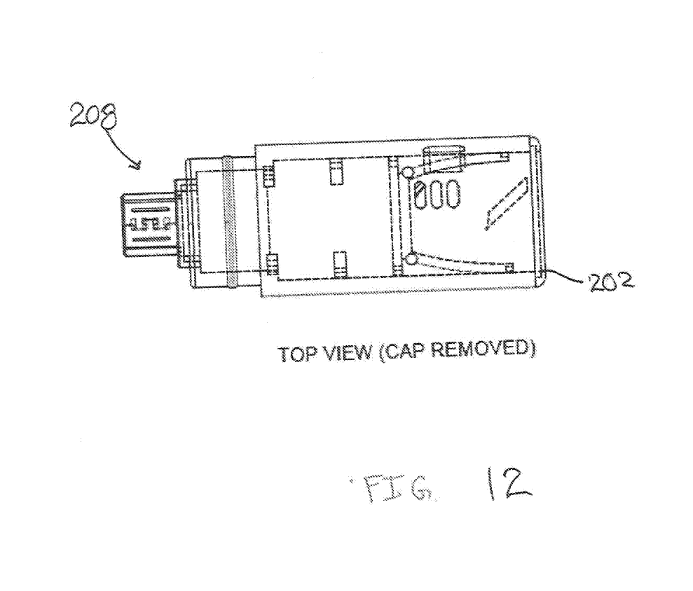

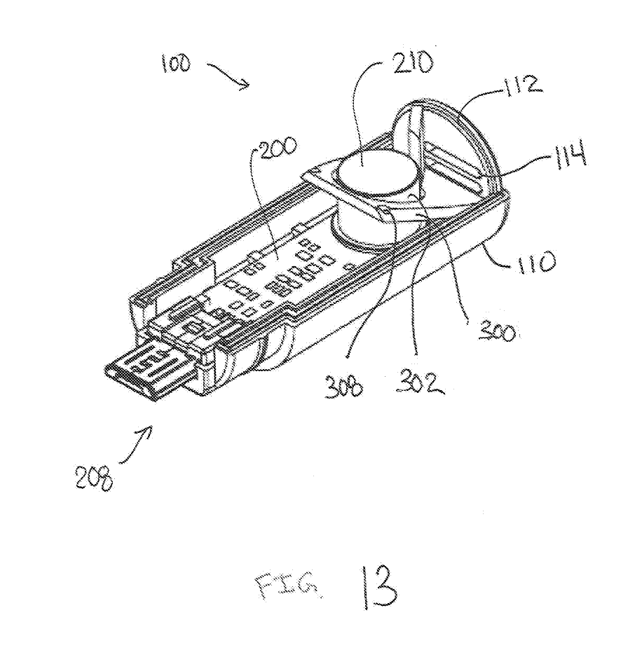

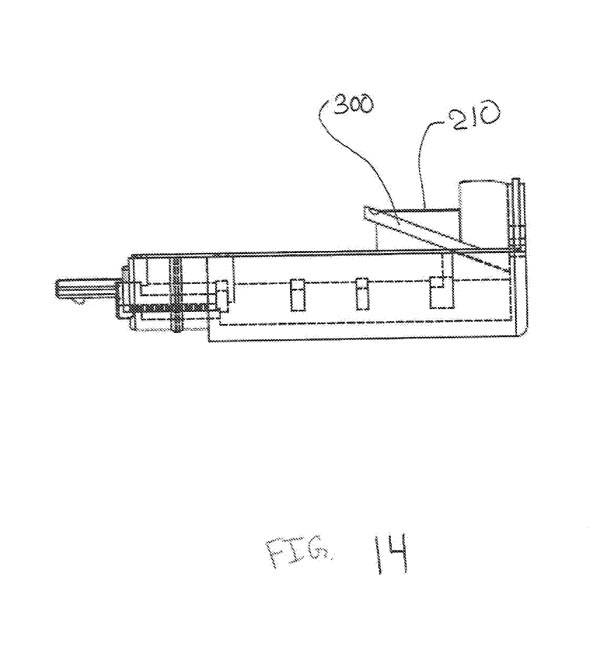

FIGS. 9-12 illustrate the device 100 of FIG. 1 with both the top half of the body portion 102 removed, and the end cap 104 removed. A more detailed representation of the connector portion 208 is illustrated in this example.

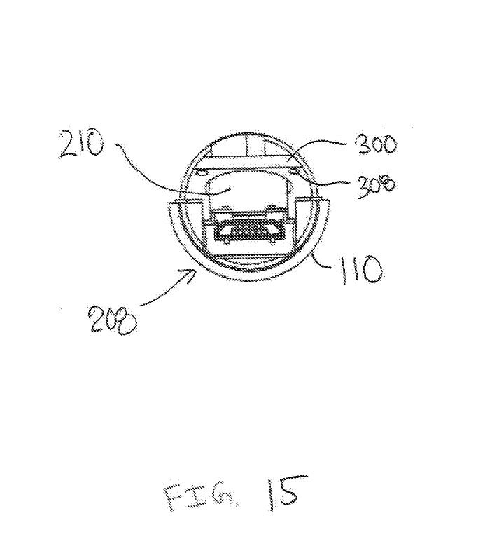

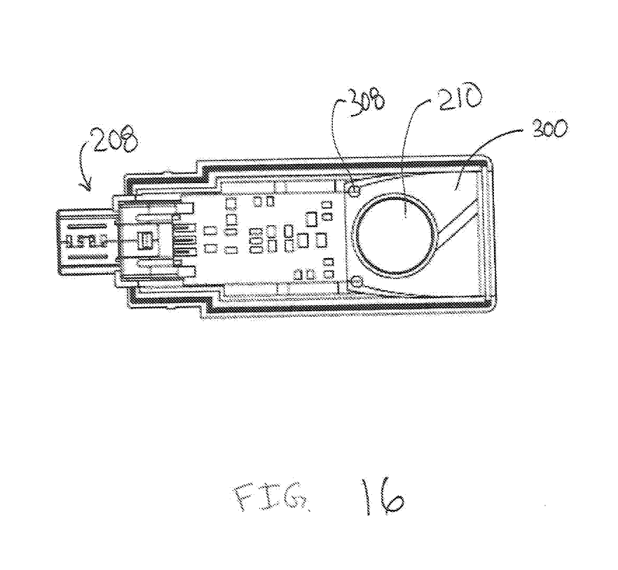

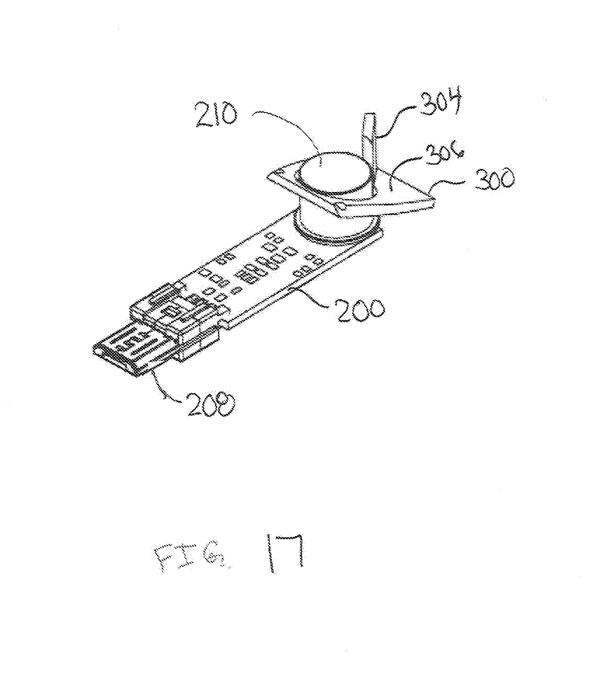

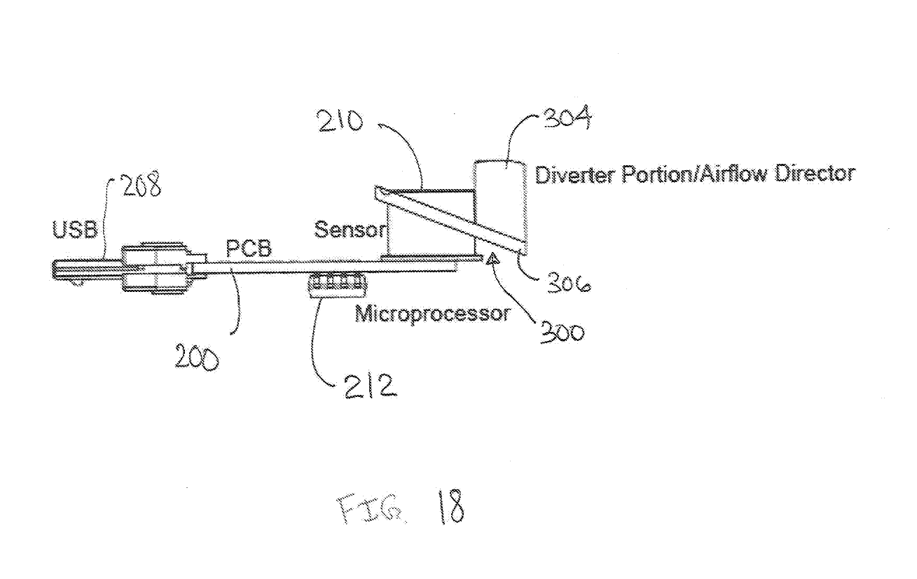

FIGS. 13-16 illustrate a perspective view of the PCB assembly 200 disposed within the bottom portion 110 of the body portion of the device 100 shown in FIG. 1. In the embodiment of FIG. 13, the PCB assembly 200 sits down below a midpoint or half of the body portion 102, within the bottom portion 110. In this example, the sensor assembly or device 302 is shown disposed on the PCB assembly 200. The sensor device 302 is generally arranged proximate the airflow device 300.

In the example shown in FIG. 13, the airflow device 300 includes an opening 212. At least a portion of the sensor device 302 is disposed within this opening 212 as it is situated on the PCB assembly 200.

As shown in FIG. 13, in one embodiment, the end 112 of the body portion 102 of the device 100 can include openings or vents 114. The vents 114 will generally be referred to as intake vents 114. The shape of the vents 114 shown in FIG. 13 are oblong. In alternate embodiments, the shape of the vents can be any suitable shape. The vents 114 are configured to allow air to impinge on the airflow device 300, as will be further described herein.

The airflow device 300 shown in FIG. 13 includes a diverter portion 304 and a ramp portion 306. The diverter portion 304 is substantially upstanding and is disposed at an angle of less than 90 degrees relative to a right edge of the ramp portion 306. The diverter portion 304 extends from near the vents 114 towards the sensor 210 disposed in the opening 302. In one embodiment, the diverter portion 304 does not extend past an edge of the sensor 200 to leave an opening or channel on one side of the interior of the body portion 102 over the ramp portion 306. The diverter portion 304 will have a height that extends from the ramp portion 306 towards the top of the sensor 210. The diverter portion 304 forms a substantial barrier within the body portion 102 and will prevent air flow from going past the diverter portion 304 into the interior of the body portion 102, except through the channel defined on one side of the ramp portion 306.

The ramp portion 306 extends at an upward angle relative to the PCB assembly 200. The ramp portion 306 extends substantially across the width of the body portion 102. As is shown in FIG. 13, for example, one end of the ramp portion 306 is substantially at the same height as, or just below, a top of the sensor 210. The ramp portion 306 is configured to force the flow of air that enters through the vents 114 in an upward direction, around and over the side of the sensor 210 and over the top of the sensor 210.

The combination of the diverter portion 304 and the ramp portion 306 forms an air flow path or channel 320. The air flow path or channel 320 runs from the intake vent 114, along a surface of the diverter 304 and upwards along the surface of the ramp 306. The channel 320 extends along a side of the sensor 210 and over a top of the sensor 210. As will explained in more detail below, the exit of the channel 320 will be the vents 106. In this manner the air flow into the device 100, the breath of the user, is caused to flow over and around the sensor 210 in a controlled manner. For example, the velocity of the airflow will be restricted or slowed as it flows along the diverter 304 and ramp 306 to the sensor 210. By controlling the velocity of airflow, the aspects of the disclosed embodiments can provide more accurate measurements and results.

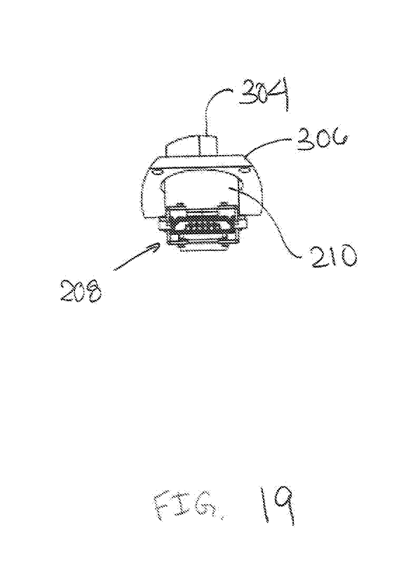

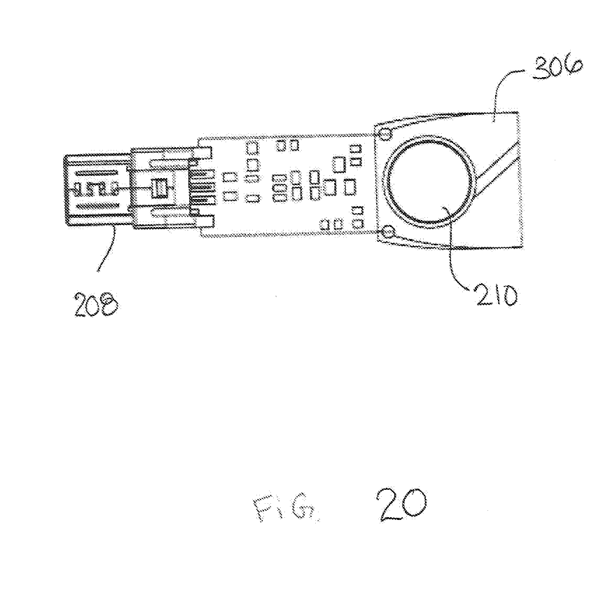

FIGS. 17-20 illustrate the PCB assembly 200 and the airflow device 300 without the body portion 102 or cap 104. In this example, the positioning of the sensor device 302 and connector portion 208 on the PCB assembly is illustrated. The airflow device 300 is also illustrated. In the example of FIG. 18, a microprocessor 212 is shown disposed on a side of the PCB assembly 200.

As is shown in FIG. 18, the ramp portion 306 is at an angle to the PCB assembly 200. In one embodiment, the angle can be between 20 to and including 60 degrees. In alternate embodiments, the ramp portion 306 can angled relative to the PCB assembly 200 at any suitable angle, other than including the range of 20 to and including 60 degrees. For example, the angle could be approximately 75 degrees. Factors affecting the angle can include the height of the sensor 210 and a distance from the end 112 of the body portion 102 to the top of the sensor 210, which are generally used to determine the length of the ramp portion 306. The diverter 304 extends upwardly relative to the PCB assembly 200, and generally can have the form of a blade or rudder, as that may be understood.

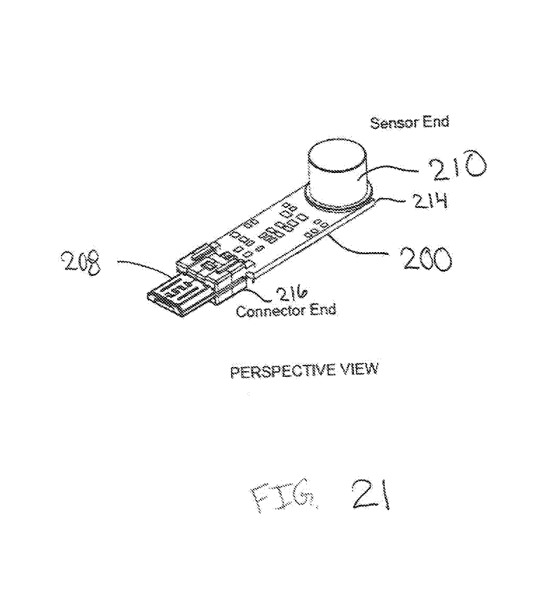

As shown in FIG. 19, in one embodiment, a forward end of the diverter 304 can be thicker or wider than the other end, the other end being closer to the end 112 of the body portion 102. This shape can be similar to that of a wing or aerodynamic blade. In alternate embodiments, the diverter 304 can have a substantially consistent thickness.

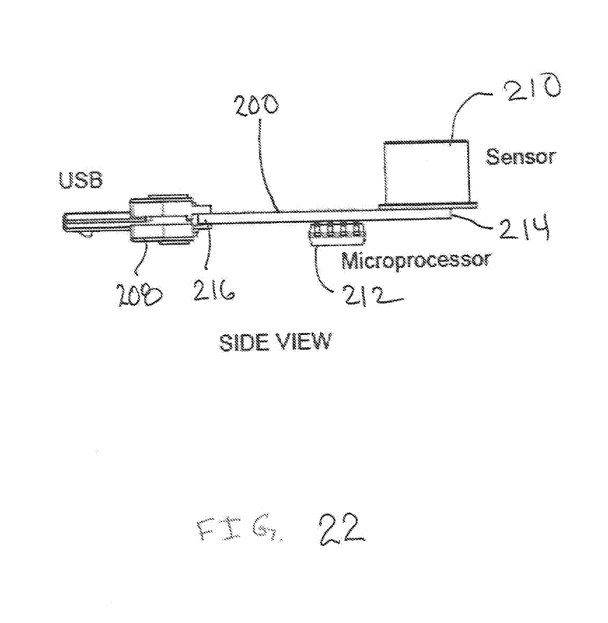

FIG. 20 illustrates one example of the air channel 320. In this example, the air channel 320 is formed and runs along the side of the sensor 210. As shown in FIG. 20, the air channel 320 is along the left side of the interior of the device 100, the side towards the bottom of the figure. The diverter 304 is positioned so that in this top down view, the end of the diverter 304 near the intake vents 114 is higher than end of the diverter 304 near the sensor 210. This deflects or diverts air entering the air channel 320 from the vents 114 to the left, in this example.

The air channel 320 runs along the left side of the diverter 304 from the inlets 114, towards a left side of the interior of the body portion. The ramp 306 disposes the air channel 320 in an upwards direction along the side of the sensor 210 and then over the top of the sensor 210. In alternate embodiments, the air channel 320 can be formed to run on either side of the sensor 210.

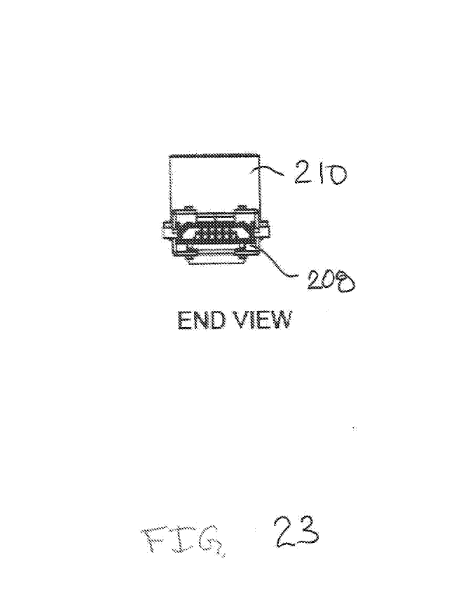

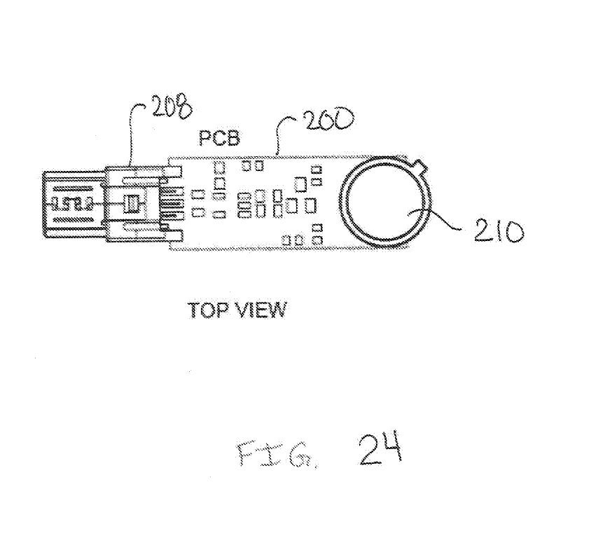

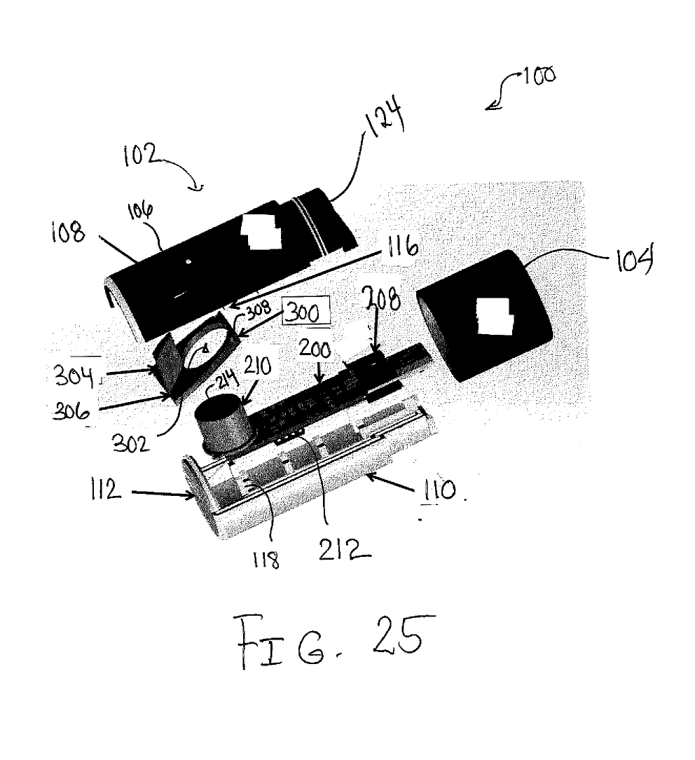

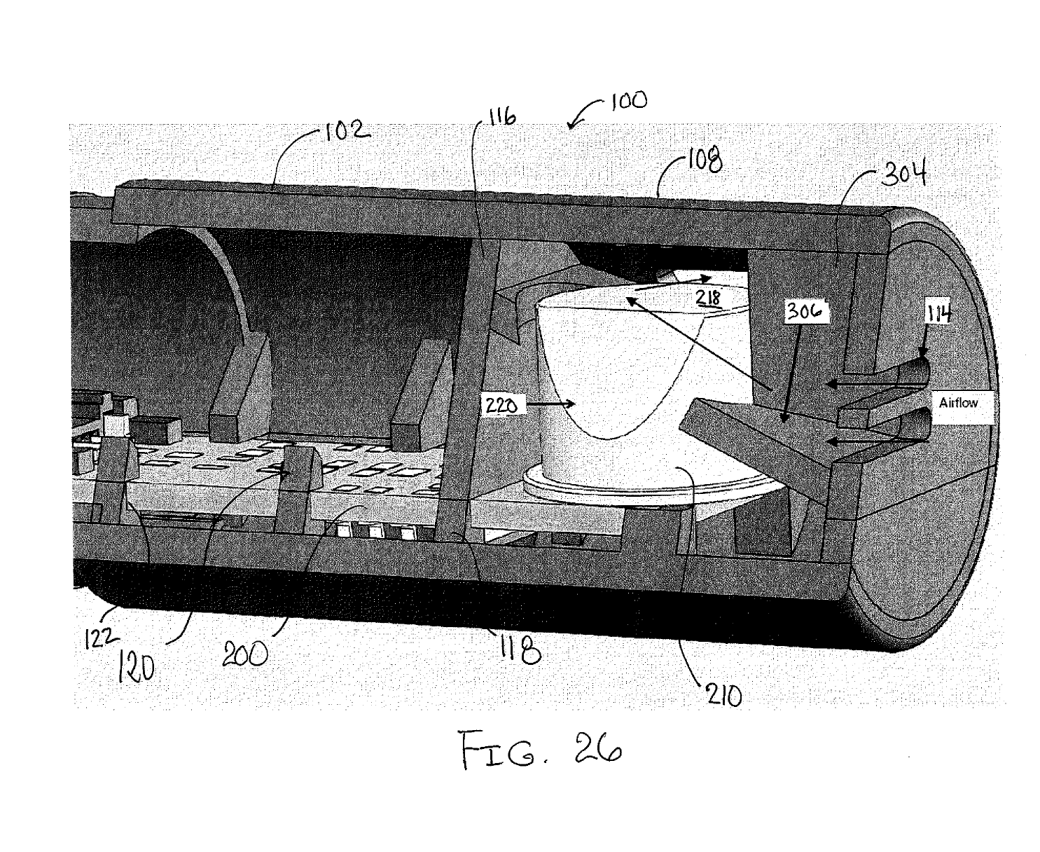

FIGS. 21-24 illustrate the PCB assembly 200. In this example, the sensor device 210 is shown at one end 214 of the PCB board or assembly 200, also referred to as the sensor end 214. The connector 208 is disposed at the other end 216 of the PCB assembly 200, also referred to as the connector end of the PCB board or assembly 200.

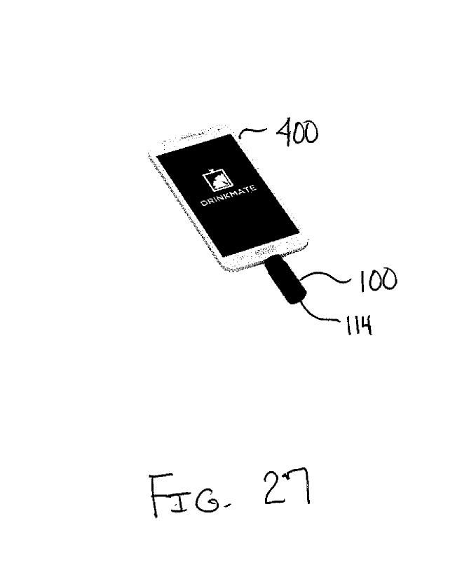

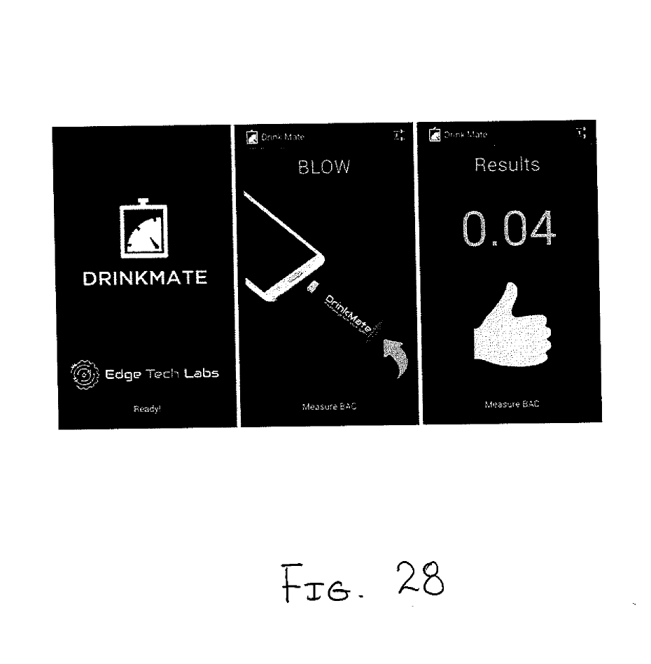

FIG. 25 illustrates an assembly view of the device 100 shown in FIG. 1. In this example, the device 100 includes the enclosure or body portion 102 and the cap portion 104. The body portion 102 includes a top portion 108, and a bottom portion 110. In the example shown in FIG. 25, the bottom portion 110 includes one or more PCB support members 118. The support members 118 are used to support and the hold the PCB assembly 200 in place within the body portion 102. A more detailed illustration of the use of the support members 118 is shown in FIG. 26. In that example, the support members 118 include snap like portions 120 that secure around a portion of the PCB assembly 200 to secure it in place.

The PCB assembly 200 includes the connector 208 on one end and the sensor 210 on the other end. The cap 104 is used to cover and protect connector 208 when not in use.

The microprocessor 212 in this example is on a side of the PCB 200 opposite the sensor 210. In alternate embodiments the microprocessor 212 can be disposed on the same side of the PCB 200 as the sensor 210.

The airflow device or assembly 300 includes the airflow director or diverter portion 304 and the ramp portion 306. The airflow device 300 is used to direct airflow that enters the interior of the body portion 102 from the vents 114 at and 112 over the sensor 210. In the example of FIG. 25, the airflow device 300 is configured to direct the air entering through the openings or vents 114 upwards and toward one side of the interior of the device 100. The aspects of the disclosed embodiments allow the airflow to be regulated and controlled to pass over a top portion 218 of the sensor 210 without being forced down onto it.

For example, in one embodiment, the user breathes into or towards the end 112 of the device 100 where the air inlets 114 are illustrated. In the example shown, the air inlets 114 comprise a pair of horizontally oriented inlets 114. In alternate embodiments, the inlets 114 can be oriented in any suitable manner, such as longitudinally or angled. The shape of the inlets 114 can be any suitable shape or side to allow air to enter.

In the example of FIGS. 13 and 26, the inlets 114 have a length in the horizontal direction that is longer than the width in the vertical direction. The inlets 114 can also be slightly off-center to the left, particularly where the air channel 320 is configured to carry the airflow along the left side of the sensor 210. In alternate embodiments, any suitable number of inlets 114 can be used in any suitable orientation, size and position.

As described generally above, the air goes through air inlets 114 and encounters the diverter portion 304 and ramp portion 306 of the airflow device 300. In the embodiment shown in FIG. 26, the ramp portion 306 forms an upwardly sloped ramp. The airflow is directed up this ramp portion 306 of the device 300. The diverter 304 is angled or has an angled portion that directs the air flowing from the inlets 114 to the let and up the ramp portion 306. In this manner the airflow enters the vents 114 and is directed around the left side 220 of the sensor 210, before flowing over the top 218 of the sensor 220. This airflow control and regulation ensures that no direct air current enters the sensor 210, which could otherwise skew results, since the sensor 210 measures ambient alcohol presence.

As shown in the example of FIG. 25, the airflow device 300 includes an opening 302. The opening 302 is configured to fit over and around the sensor 210. This opening 302 can be used to position the airflow device 300 within the interior of the device 100. In one embodiment, the airflow device 300 is secured within the interior portion of the device 100. For example, in one embodiment, the airflow device 300 is configured to be snapped or secured into position after the PCB assembly 200 with the sensor 210 is disposed within the bottom portion 110 of the body portion 102.

Referring also to FIG. 1, the top or upper portion 108 of the body 102 includes openings or vents 106. The vents 106 are also referred to herein as exhaust ports. In one embodiment, the exhaust vents 106 can be provided one on either side of the enclosure, only one of which is shown for air to leave the area of the sensor 110 and ventilate after use.

An example of the exemplary airflow is shown in FIG. 26. After flowing over the sensor 210, the airflow exits the interior of the device 100 through the exhaust vents 106. The exhaust vents 106 for the exhaust portion of the air channel 320.

Referring to FIGS. 25 and 26, in one embodiment, the body portion 102 includes a rib or wall member 116 that is disposed just in front of the sensor 210 in an assembled state of the device 100. In the example of FIG. 26, the rib member 116 mates with the PCB support member 118 on the side of the sensor 210 that is away from the vents 114. The rib member 116 extends across the interior of the body portion 102, from one side to another and forms a wall that blocks or prevents the airflow from moving further into the interior of the body portion 102. The rib member 116 advantageously prevents humid human breath from moving further forward over the PCB assembly 200 to where there are other electrical components.

The PCB assembly 200 includes all of the electrical components including the connector 208, the sensor 210 and the microprocessor 212. The connector 208 is used to plug the device 100 into mobile devices, such as smart phones. The sensor 210 is used to sense and detect blood alcohol in an airflow, as is generally understood. In this example, the sensor 210 can include any suitable blood alcohol sensor that can be used in conjunction with the aspects of the disclosed embodiments. The microprocessor 212 is configured to use the sensed blood alcohol to determine a blood alcohol level and output that data onto a screen of the connected mobile device.

The PCB 200 also includes suitable electronic circuitry. The primary components of the circuit of the PCB assembly 200 can include, but are not limited to, the alcohol (ethanol) semiconductor sensor 210; a micro-USB connector 208; an adjustable voltage regulator (not shown); a MOSFET (transistor)(not shown) and a Microcontroller (not shown). In alternate embodiments, the PCB assembly 200 of the device 100 can include any other suitable or needed components in any suitable positions or locations on the PCB assembly 200.

Some of the key features provided by the PCB assembly 200 of the disclosed embodiments include:

USB or iAP2 communication without a separate hardware chip. Only the microcontroller is used to communicate with the mobile device.

Power supplied to the sensor 210 can be adjusted remotely (from the mobile device) according to its pre-heating needs (utilizes the MOSFET/transistor).

The PCB assembly 200 can be powered entirely via the connection to the mobile device.

Referring again to FIGS. 25 and 26, bottom portion 110 of enclosure 102 includes notches 122. The notches 122 are used in conjunction with the snap like portions 120 described above to hold the PCB assembly 200 tightly in place.

The connector 208 is configured to mate with and connect to a corresponding connector in a mobile device, such as a smartphone. In the example shown in FIG. 25, the connector 208 is a USB type connector. In alternate embodiments, any suitable connector can be used, such as a Lightning connector.

In one embodiment, the assembly of the device 100 includes the following:

The airflow device 300 is pushed up into the upper portion 108. The upper portion 108 can include posts (not shown) that engage corresponding openings 308 in the ramp portion 306. The airflow device 300 is held in place by the friction of the two posts in openings 308.

The PCB assembly 200 is pushed down into the bottom portion 110. The notches 122 and snap devices 120 hold the PCB assembly 200 in place.

The upper portion 108 and the lower portion 110 are mated together. In one embodiment, there are snaps on lower portion 110 that the upper portion 108 clips into upon being pushed together. This connection is meant to be secure, and in some cases permanent.

The cap 104 is pushed onto the body portion 102 over and around the connector 208. The cap 104 is used to cover the connector 208.

The PCB board assembly 200 is typically assembled using a pick-and-place machine that is used for all surface mount components. The “hand-solder” components (connector 208 and cylindrical sensor 210) are then soldered on. In the embodiment shown, the USB connector assembly 208 is “mid-mount” and straddles the PCB assembly 200. There are solder connections on both sides of the PCB assembly 200. This relieves pressure on the solder joints when the device 100 is pushed into or coupled to a mobile device.

Similarly, when the device 100 is pulled out or decoupled from a mobile device, the front 124 of the enclosure 102 pushes against the corresponding front of the connector 208 instead of directly on the solder joints. In this way, the connector 208 will have less stress on its solder connections over the course of its life. Other embodiments may utilize a different method for securing the connector 208 to the enclosure 102 and connecting it to the PCB assembly 200.

As noted above, the device 100 of the disclosed embodiments can be made in a much smaller size or package, primarily due to the elimination of the need for a battery. Power is supplied to the device 100 by the host mobile device, such as a smartphone, using for example, USB hosting.

The device 100 of the disclosed embodiments does not require a “mouthpiece”, as might otherwise be understood. The air inlet assembly 114 and the airflow device 300, described above can reduce the speed of the airflow of the introduced air (the person breathing on or blowing on or at the inlet area) to a more standardized velocity prior to measurement. This provides reliable and repeatable results.

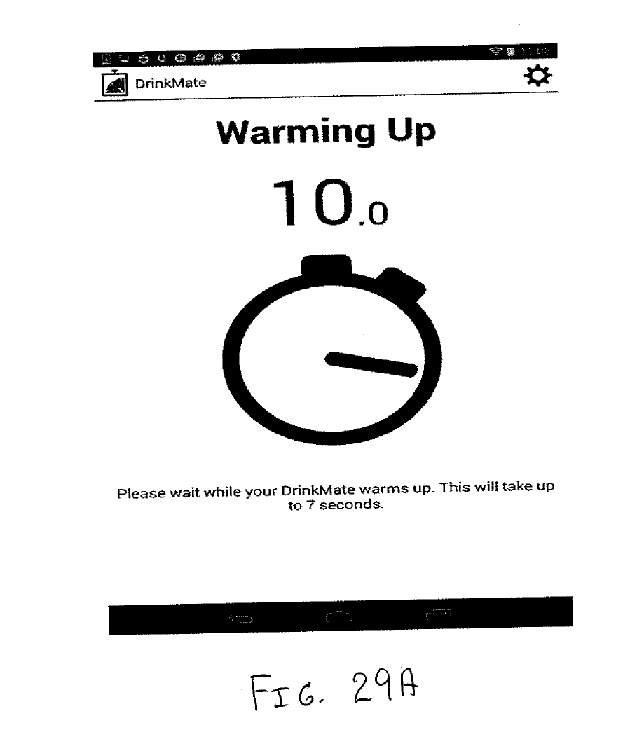

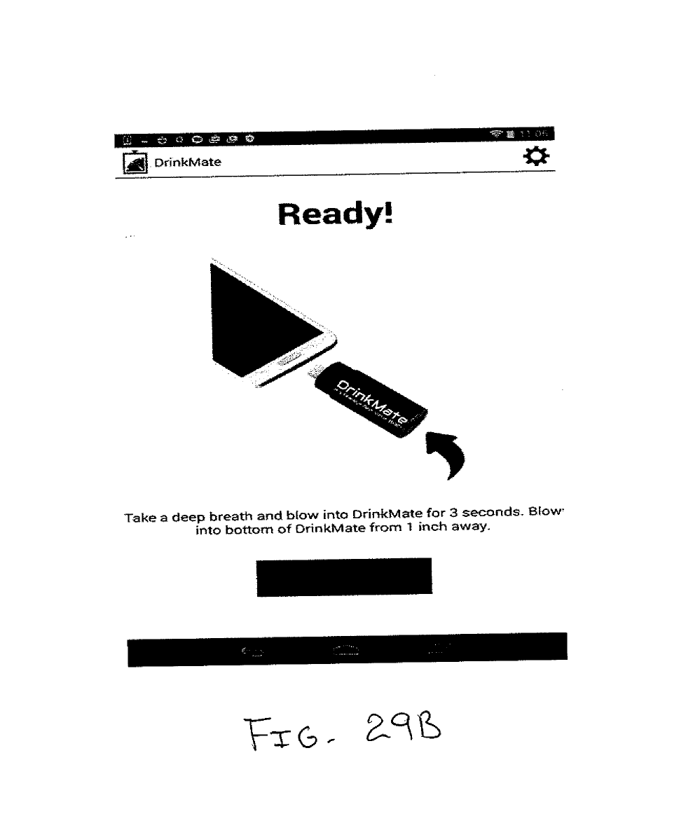

Referring to FIGS. 27 and 28, in one embodiment, the breathalyzer device 100 of the disclosed embodiments, is connected or “plugged” into a smartphone device 400. The device 100 is held securely in place when plugged into the smartphone 400 or other device using an ultra high-quality micro-USB connector or Apple Lightning connector. The connector 208 is configured to couple the device 100 to the computing device 400. Although a USB style connector is referred to herein, in alternate embodiments any suitable connector or connection can be used, including for example, wireless coupling.

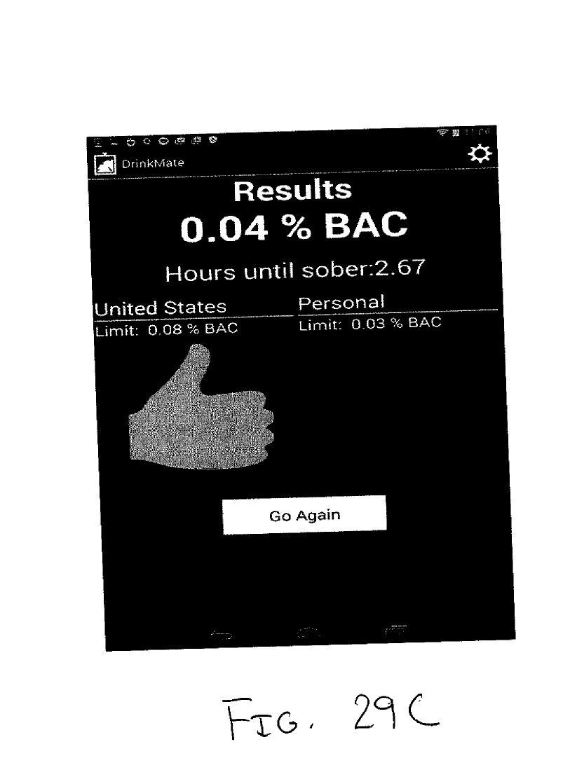

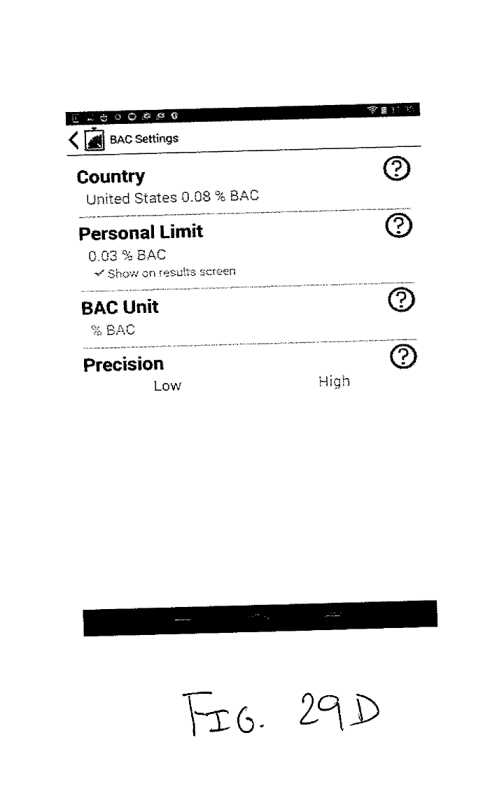

In one embodiment, the smartphone device 400 will be enabled with o include a corresponding software application. The application provides the necessary interface between the device 100 and the smartphone device 400. Referring to FIGS. 29A-29D, once the application is downloaded or otherwise stored on the smartphone device 400, upon plugging the device 100 into the smartphone device 400, the application can automatically control the following:

Receives power;

Establishes a communication connection;

Automatically recognizes the device 100 and opens the DrinkMate application;

Instructs the user to wait until the DrinkMate device 100 is warmed up. This is a “pre-heat” process for an adjustable number of seconds to warm up the sensor 210.

After pre-heated, the device 100 goes into a steady state where no power adjustments are made and the user's breath alcohol measurements are made.

Provides the user instructions on how to properly take a breath alcohol measurement.

A person will blow onto or into the sensing area of the device 100, generally defined by intake vents or openings 114 in the end 112 of the device 100. The BAC is measured and the results presented on a display of the device 400.

In one embodiment, the device 100 will send the measured BAC data to the smartphone device 400 via the physical connector 208 such as for example a micro-USB connector, Apple Lightning connector, USB Type C connector, or any other industry-standard connector. After blowing, the BAC calculation takes a fraction of a second due to the smartphone's powerful processor and is substantially immediately displayed or otherwise presented to the user, as is generally shown in the sequence of exemplary screen shots shown in FIGS. 28, 29A and 29C.

Advantageously, the device 100 of the disclosed embodiments does not need an internal battery or power supply. Rather, the device 100 receives power from the smartphone device 400. In one embodiment, power consumption ranges from about 30 mA for steady state to 100 mA during warm up, which is only about 7 seconds. This consumption is generally negligible for short periods of time on any phone or smartphone device.

While a smartphone device is referred to herein, the aspects of the disclosed embodiments are not so limited and the reference to a smartphone device can generally include any mobile computing or communication device, such as mobile telephones, tablets, pads, phablets, smart computing devices and other mobile communication and computing devices generally.

The device 100 of the disclosed embodiments incorporates a low-cost advanced stability semiconductor-ramped sensor 210 for measuring the BAC. As described herein, the device 100 includes innovative air inlets 114 and an airflow device 300 that direct air flow over the sensor 210 such that readings are precise and repeatable. The air inlets 114 and airflow device 300 of the device 100 also work to slow airflow that is too fast, which allows for a greater range of breath air speed.

The air outlets 106 of the device 100 are positioned so that alcohol can quickly clear the sensor area once a reading is taken.

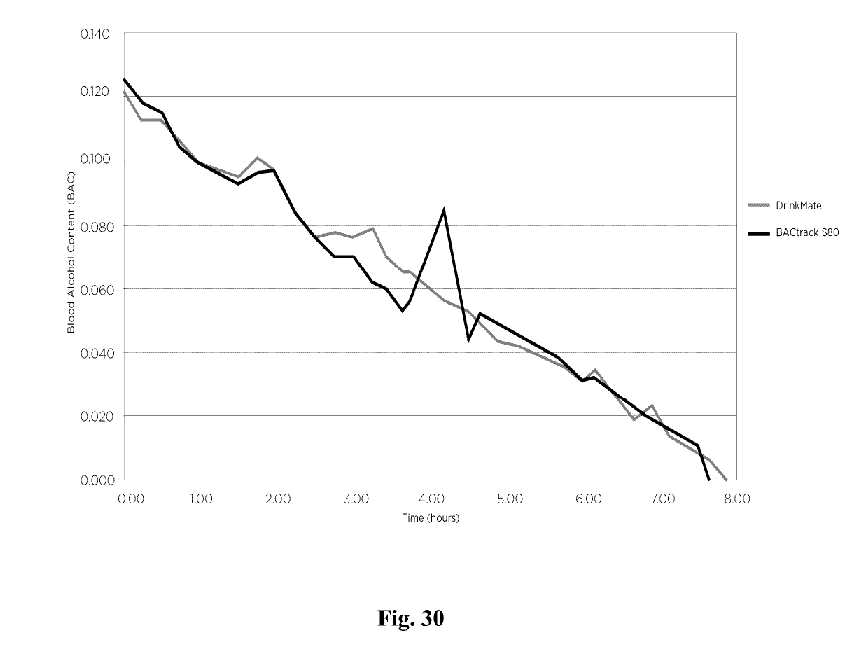

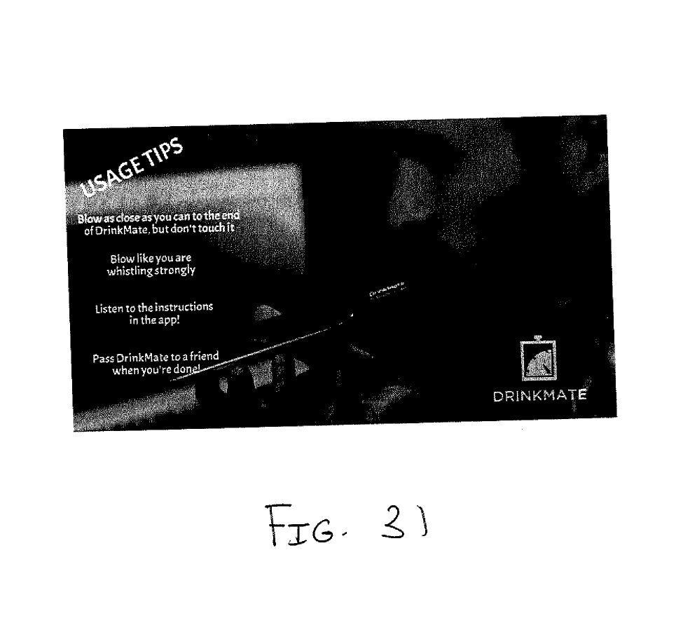

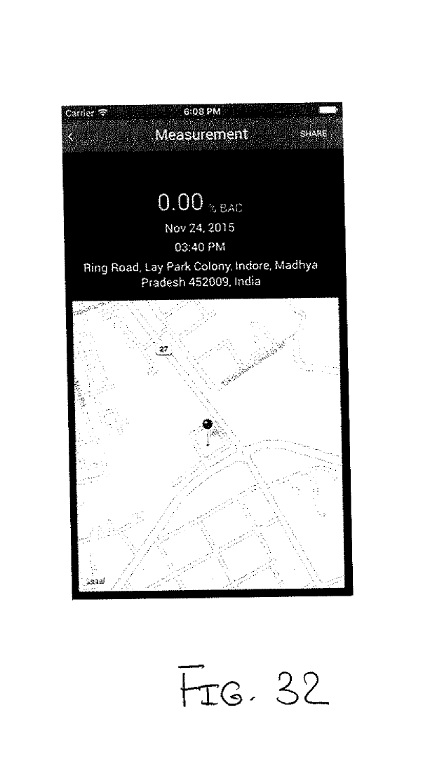

Accuracy is approximately +/−0.01% BAC at a BAC of 0.02%. The article attached as FIG. 30 describes aspects of the accuracy and performance of the device 100 described herein.

In one embodiment, maximum BAC of the sensor 210 can be limited to approximately 0.20% BAC. In alternate embodiments, any suitable limit can be imposed, or none at all.

The accuracy of the device 100 of the disclosed embodiments was validated using testing and calibration kits from Lifeloc Technologies, the leader in breathalyzer testing and calibration.

Most of the weight in portable electronics comes from the batteries that are used to power the device. Thus, in one embodiment, the device 100 of the disclosed embodiments does not include a battery. Rather, the device 100 derives the power needed to operate the device 100 from the smart phone or other computing device to which it is connected. While the aspects of the disclosed embodiments are generally described herein as not including a battery, in alternate embodiments a battery or other power supply can be included. This can include small light weight batteries, or wirelessly powered devices or power supplies.

The algorithm used in the device 100 of the disclosed embodiments accounts for sensor changes over time and during first uses. For the BAC calculation algorithm, the aspects of the disclosed embodiments chemically characterize how the sensor 210 measures alcohol and the algorithm adjusts accordingly to certain measured characteristics. In one embodiment, the sensor 210 measures ethanol (alcohol) using a tubular ceramic element covering a tin dioxide core. As ethanol is exposed to the exterior, the electrical resistance of the system changes. This change is measured, which varies based on the amount of ethanol in the air to which it exposed to.

The screenshots of FIGS. 29A-29D and 32 are exemplary screen shots of the user or application interface for the DrinkMate device 100 of the disclosed embodiments. The different screens can provide general instructions as to the operation of the DrinkMate device and the user interaction with the DrinkMate device, as well as present the results.

FIG. 29D illustrates a settings page, where the user can adjust the various parameters, limits, units and precision of the device 100. The layout, style and number of application pages, or screen shots shown herein are merely exemplary and are not intended to be limiting to the scope of the disclosed embodiments. In alternate embodiments, the various information, settings and results can be presented in any suitable manner on any number of screens or pages.

In summary, some of the key aspects of the device 100 and process of the disclosed embodiments include:

No battery requirement. Power is supplied entirely by the mobile device.

Inlet design reduces sensor variations due to breath air velocity differences by slowing the air down and redirecting it in a consistent manner.

No mouthpieces required. Users do not need to put their mouth on the device. This helps to reduce the spread of diseases when shared.

No recalibrations necessary. The algorithm (stored and updated on the mobile device) accounts for sensor variations and changes over time. The sensor characteristics can be stored in a controller of the device 100, such as the micro-controllers EEPROM. The DrinkMate device 100 of the disclosed embodiments is not dependent upon its application for individual sensor data. The device 100 of the disclosed embodiments measures certain sensor characteristics and adjusts the BAC output accordingly to continue registering and accurate and precise result. This also significantly increases the life of the device.

Thus, while there have been shown, described and pointed out, fundamental novel features of the invention as applied to the exemplary embodiments thereof, it will be understood that various omissions, substitutions and changes in the form and details of devices and methods illustrated, and in their operation, may be made by those skilled in the art without departing from the spirit and scope of the invention. Further, it is expressly intended that all combinations of those elements, which perform substantially the same function in substantially the same way to achieve the same results, are within the scope of the invention. Moreover, it should be recognized that structures and/or elements shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

What is claimed is:

1. An portable blood alcohol sensing device comprising:
an enclosure having an end with at least one vent opening defined therein;
a printed circuit board assembly disposed within the enclosure, the printed circuit having a connector at one end and a sensing device at an other end;
an air flow device disposed within the enclosure adjacent to the at least one vent opening, the air flow device including a ramp portion and a diverter portion, the ramp portion including an opening, a body portion of the sensor configured to be disposed within the opening in the ramp portion;
wherein the ramp portion is disposed substantially horizontally at an upward angle relative to the printed circuit board assembly and the diverter portion is disposed substantially longitudinally and an inward angle relative to the at least one vent opening;
the at least intake vent opening, the diverter portion and the ramp portion defining an airflow path through an interior of the enclosure, along a side and over a top portion of the sensing device.

2. The device of claim 1, wherein an airflow through the intake vent openings is directed by the diverter portion to a side of the diverter and onto the ramp portion, the ramp portion directing the airflow along the side of the sensing device and over the top of the sensing device.

3. The device of claim 2, comprising one or more exhaust vent openings defined in a top portion of the enclosure, the one or more exhaust vent openings being disposed above the top of the sensing device.

4. The device of claim 3, wherein the airflow in the airflow path exits the interior of the enclosure through the exhaust vent openings.
